(19) United States
(12) Patent Application Publication
(10) Pub. No.: US 2019/0309249 A1
(43) Pub. Date: Oct. 10, 2019

(54) TRAY FOR SUPPORTING INDIVIDUAL OR MULTIPLE CELL CULTURE WELLS

(71) Applicant: Corning Incorporated, Corning, NY (US)

(72) Inventors: Sheila Anne Carvalho, Mattapoisett, MA (US); William Joseph Lacey, North Andover, MA (US)

(21) Appl. No.: 16/463,168

(22) PCT Filed: Nov. 29, 2017
(86) PCT No.: PCT/US2017/063567

§ 371 Date: May 22, 2019

Related U.S. Application Data
(60) Provisional application No. 62/428,144, filed on Nov. 30, 2016.

(51) Int. Cl. C12M 1/12; C12M 1/32

(52) U.S. Cl. CPC C12M 25/04; C12M 23/12

(57) ABSTRACT

The disclosure provides a cell culture well tray assembly having a reservoir plate for containing cell culture media, a removable tray structured to fit on top of the reservoir plate, having a top surface and an opposing bottom surface defining a deck, and a plurality of well apertures in the deck, at least one removable well, structured to fit through a well aperture and extend below the bottom surface of the deck of the tray, each well having a well enclosure for containing cell culture material and a hanger, wherein when the removable well is inserted into a well aperture, there is a space between the deck of the tray and the hanger of the well.

### CROSS-REFERENCE TO RELATED APPLICATIONS

This application claims the benefit of priority of U.S. Provisional Application Ser. No. 62/428,144 filed on Nov. 30, 2016 the content of which is relied upon and incorporated herein by reference in its entirety.

### FIELD

The present disclosure relates generally to cell culture containers useful for growing cells for randomized screening. The disclosure provides a tray structured to contain and support removable cell culture wells in a reservoir containing cell culture media. The tray has features, such as raised well walls or collars or angled well openings or well walls to provide a space between the deck of the tray and the removable well, to allow the cell culture wells to be individually introduced into or removed from the tray. The tray may also have feet to allow the contained and supported cell culture wells to be suspended over and partially immersed in the reservoir of cell culture media, to stack one tray on another, or to support the tray on a surface.

### BACKGROUND

Cells in culture are used for many purposes. Cells in culture are used to test the effectiveness of new chemical entities by testing how new chemical entities affect physiological responses in cells. Often, these tests are performed early in the development of drugs. High throughput screening can be employed to allow testing of large numbers of drugs or potential drugs on a population of cells. Often, these screenings are performed in multi-well plates. Plates may have 6, 12, 24, 96, 384 or 1526 wells, for example, each containing a population of cells. These cells can be grown in many configurations, including isolated populations of cells, in 2D (cells grown as a monolayer) where the cells adhere to the surfaces of a cell culture vessel, in 3D (cells grown as spheroid bodies) where cells grow in suspension, or in co-culture conditions where multiple cell types are grown together in culture, to name a few. Cell culture vessels can be configured to support specific cell culture needs, depending upon the specific needs of the user. Traditional multi-well plates are plates having integral wells. Removable wells which can sit inside integral wells are known. For example, cell culture inserts are available from Corning Incorporated (Transwells® and Netwells®), from Millipore (Millicell®), from Greiner (ThinCert™) and from Nunc (polycarbonate membrane inserts in multi dishes and carrier plates). These products are available as individual well inserts or as multi-well inserts containing multiple integral wells designed to insert into standard 6, 12, 24 or 96 well plates or into a shared reservoir. A Netwell® carrier is available from Corning Incorporated which provides a support with a handle that can be used to manually exchange individual wells into and out of the carrier as well as provide multiple individual wells to a standard multi-well plate. However, there is a need for improved carriers or trays that can be used to support, transport, manipulate, exchange, and randomize individual wells, introduce those wells to a reservoir, remove the wells from the tray and the reservoir, whether that reservoir is a shared reservoir or a multi-well plate reservoir, and conduct studies on the contents of these individual wells.

### BRIEF SUMMARY

In embodiments, a cell culture tray which allows individual wells of a multi-well cell culture vessel to be removed and exchanged, introduced into a reservoir containing cell culture media or other fluid, removed from the reservoir and placed on a surface is provided. The disclosure provides a tray to contain and support removable cell culture wells, the tray having feet to allow the contained and supported cell culture wells to be suspended over or partially immersed in a reservoir of cell culture media. The tray can also be placed on a surface. The tray allows the cell culture wells to be manipulated individually or as a group, as the wells are introduced into or removed from a cell culture media reservoir.

In an aspect (1), the disclosure provides a cell culture well tray assembly having a reservoir plate for containing cell culture media; a removable tray structured to fit on top of the plate, having a top surface and an opposing bottom surface defining a deck, and a plurality of well apertures in the deck; and at least one removable well, structured to fit through a well aperture and extend below the bottom surface of the deck of the tray, each well having a well enclosure for containing cell culture material and a hanger; wherein when the removable well is inserted into a well aperture, there is a space between the deck of the tray and the hanger of the well. In embodiments, the cell culture well tray assembly of claim 1 wherein each well aperture comprises a raised rim extending upwardly from the top surface of the deck. In an aspect (2), the disclosure provides the cell culture well tray assembly of aspect 1 wherein each well aperture comprises a raised rim extending upwardly from the top surface of the deck. In an aspect (3), the disclosure provides the cell culture well tray assembly of aspect 1 or 2 wherein the removable well comprises an angled sidewall which catches against a well aperture to create a space between the hanger of the well and the deck of the tray. In an aspect (4), the disclosure provides the cell culture well tray assembly of any one of aspects 1-3 wherein each aperture further comprises a seat and wherein the removable well comprises a tall side wall whose height is greater than the depth from the top of the seat to the top surface of the deck. In an aspect (5), the disclosure provides the cell culture well tray assembly of aspect 2 wherein the raised rim or the well or both comprise a lock feature to releasably lock the well in place in the aperture. In an aspect (6), the disclosure provides the cell culture well tray assembly of any one of aspects 1-5 wherein the tray further comprises a plurality of feet extending from the opposing bottom surface of the deck wherein the feet are structured to insert between wells of a multi-well plate. In an aspect (7), the disclosure provides the cell culture well tray assembly of any one of aspects 1-6 further comprising at least one fill aperture in the deck. In an aspect (8), the disclosure provides the cell culture well tray assembly of aspect 7 wherein the hanger comprises a lock feature. In an aspect (9), the disclosure provides the cell culture well tray assembly of any one of aspects 1-8 wherein the reservoir plate is structured to receive at least a portion of the at least one well extending below the bottom surface of the deck of the tray. In an aspect (10), the disclosure provides the cell culture well tray assembly of aspect 9 wherein the reservoir plate comprises a single reservoir. In an aspect (11), the disclosure provides the cell culture well tray assembly of aspect 9 wherein the reservoir plate is a multi-well plate, each well of the multi-well plate structured to individually contain media and a cell culture well. In an aspect (12), the disclosure provides the cell culture well tray assembly of any one of aspects 1-11 further comprising a lid structured to fit on the tray. In an aspect (13), the disclosure provides the cell culture well tray assembly of any one of aspects 1-12 wherein the tray further comprises a raised bezel around the outer periphery of the deck. In an aspect (14), the disclosure provides the cell culture well tray assembly of any one of aspects 1-13 wherein the removable well comprises a well bottom inner surface which comprises a treatment. In an aspect (15), the disclosure provides the cell culture well tray assembly of any aspect 14 wherein the treatment comprises gas permeable membrane, porous membrane, cell adherent treatment, cell non-binding treatment, micro-cavities, or a combination.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 is a perspective view of an embodiment of a tray;

FIG. 2A is a side perspective view of an embodiment of a tray and a well;

FIG. 2B shows an embodiment of a well;

FIGS. 3A, 3B, 3C, 3D and 3E are cross-sectional side views of embodiments of wells engaged in well apertures of trays;

FIG. 4 is perspective view showing locking features in the well aperture and the well sidewall, in embodiments;

FIG. 5 is a perspective view of an embodiment of a tray containing removable wells;

FIG. 6 is an exploded view of a lid, removable wells, a tray and a reservoir according to embodiments;

FIG. 7A is a side view of two stacked trays, according to an embodiment; FIG. 7B is a side view of a tray inserted into a multi-well plate reservoir, according to an embodiment; and,

FIG. 8 is a cut-away side view n exploded view of a lid, a well, a tray and a multi-well plate reservoir, according to embodiments.

### DETAILED DESCRIPTION

Reference will now be made in greater detail to various embodiments of the subject matter of the present disclosure, some embodiments of which are illustrated in the accompanying drawings. The same reference numerals will be used throughout the drawings to refer to the same or similar parts.

In embodiments, the present disclosure provides a tray, which can be removably inserted into a cell culture reservoir, which in turn holds individual removable wells. The presence of the tray provides significant advantages. For example, individual wells may be removed from the tray, and rearranged so that a user can organize or randomize samples found in individual wells. For example, in a 6-well tray (where the wells are organized in rows A and B and columns 1, 2 and 3), engaged with a 6-well multi-well plate reservoir, a user may wish to exchange the sample in well A1 with the sample in well B3, in order to organize or re-organize samples for further testing. Because each well is removable, the user can re-organize the samples, within the same tray or between trays. In addition, because the tray itself is removable from the reservoir, a user can remove a 6-well tray, containing 6 samples, and place all 6 samples into a different reservoir, which may contain different media, or different experimental conditions. Of course, the tray may accommodate a single well or multiple wells. For example, the tray may accommodate 1, 2, 3, 4, 5, 6, 12, 24, 96, 384 or 1536 wells, each of which may be individually inserted into the tray or removed from the tray. In embodiments, the removable tray and the removable wells have features that allow for easy removal and insertion of the wells into the trays, as described below. In embodiments, the tray and the wells have features that provide a space between the top surface or deck of the tray, and a rim or flange or hanger of the well to provide a user the ability to grasp wells using tools such as forceps

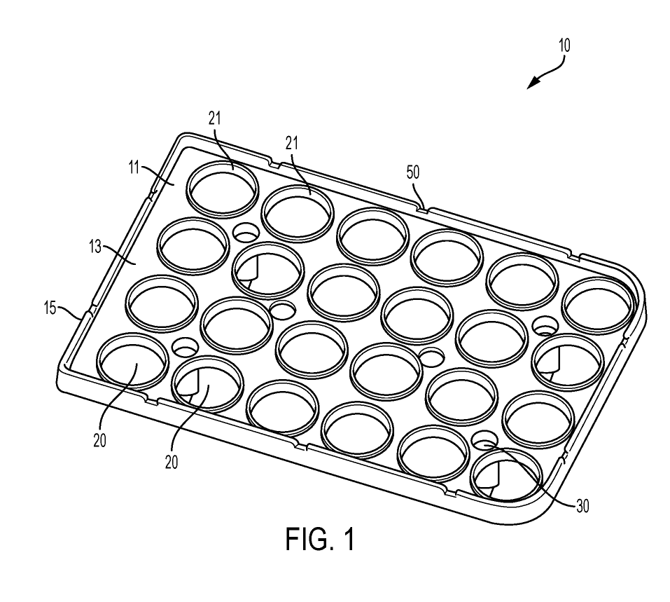

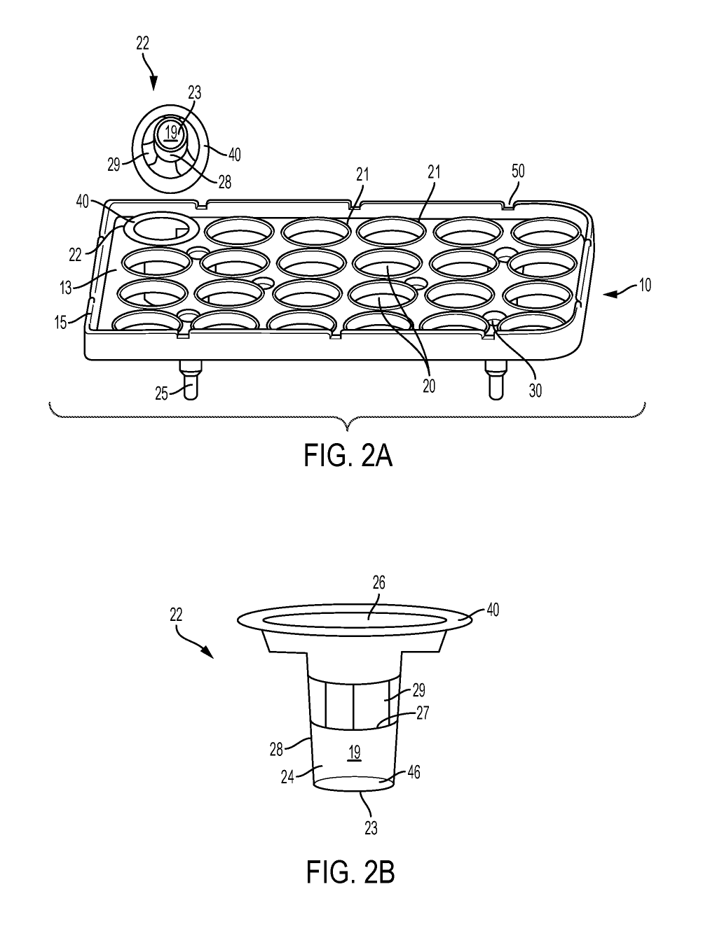

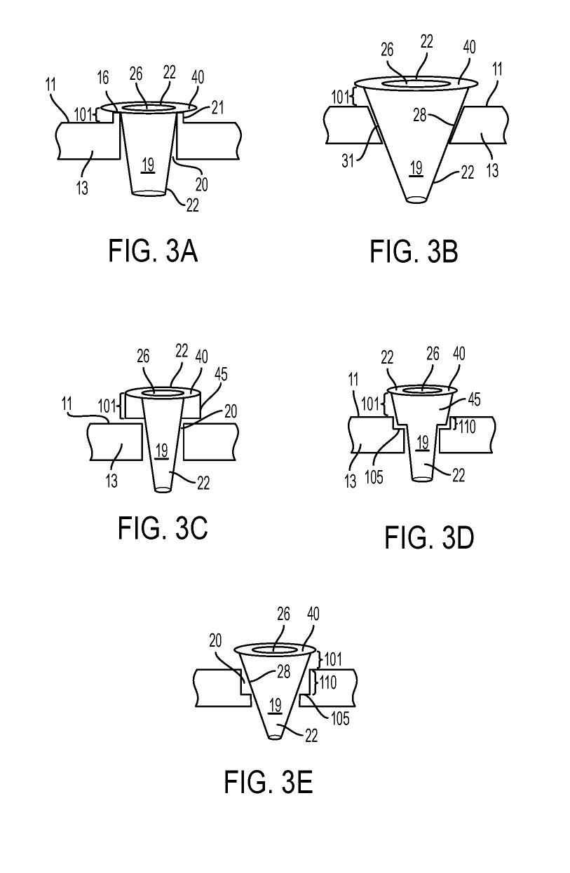

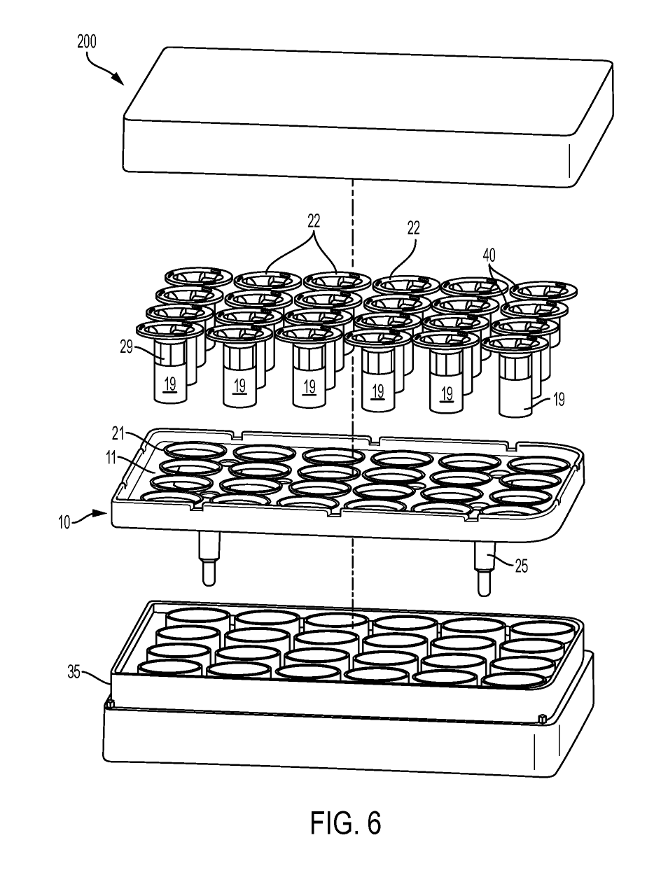

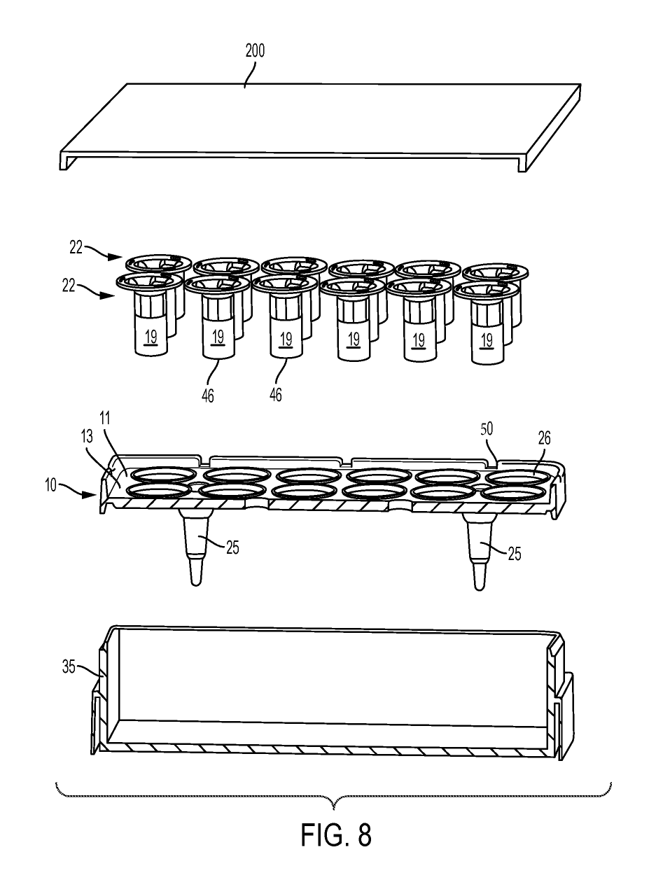

FIG. 1 is a perspective view of an embodiment of a tray 10 having a top surface 11, an opposing bottom surface 12 (not shown in FIG. 1, but see, for example, FIG. 6) defining a deck 13, and a raised bezel 15 extending from the top surface 11 at the periphery of the deck 13. In embodiments, the raised bezel 15 provides rigidity and structure to the tray, and also provides surface area at the periphery of the deck to enable robotic plate handlers to grip and manipulate the tray 10. In embodiments, the raised bezel 15 may completely encircle the deck (as shown in FIG. 1), or in embodiments, the raised bezel 15 may be present only on two sides of the deck 13, or may be discontinuous around the periphery of the deck 13. Raised bezel 15 may have notches 50. FIG. 1 also shows a plurality of well apertures 20. In embodiments, the tray 10 may have one or more well apertures 20. In embodiments, the tray 10 may have 1, 2, 3, 6, 12, 24, 96, 384 or 1536 well apertures 20 to engage with standard 1, 2, 3 6, 12, 24, 96, 384 or 1536-well multi-well plates. The well apertures 20 are structured to accommodate or hold wells 22. (See, for example, FIG. 2 or FIG. 3A-3E.) In embodiments, each well 22 has a well enclosure 19 for containing cell culture material 106 and a hanger 40. Wells 22 can be placed into well apertures 20. In embodiments, wells 22 are removable from the tray 10. In embodiments, the wells 22 engage with the well apertures 20, and are held in place in the tray 10 by the well apertures 20, and are suspended over, lowered into or are placed into cell culture media contained in a cell culture media reservoir 35 (see, for example, FIG. 6 and FIG. 8). In embodiments, the cell culture media reservoir 35 can be a multi-well plate, having multiple reservoirs, or a single reservoir plate having a single reservoir. In embodiments, the removable tray and the removable wells have features that allow for easy removal and insertion of the wells into the trays, as described below.

FIG. 1 also shows a notch 50 in the raised bezel 15. Notch 50 may or may not be present, in embodiments. Notch 50 corresponds to small ribs or gussets that may be present on or in the inner surface of a lid, allowing the lid to fit into place on the tray 10 in a way that reduces lateral movement of the lid against the tray 10. Additionally, notch 50 may allow the lid to sit down closer to the tops of wells 22 when wells 22 are in place in the tray 10. This may act to reduce evaporation from the wells 22 as well as create less opportunity for contamination of the contents of the wells 22.

Turning now to FIG. 2A, a side perspective view of an embodiment of a tray 10 is provided. FIG. 2A shows a tray 10 with a well 22 having a hanger 40 inserted into one well aperture 20 of the tray 10, and a second separate well 22 ready to be inserted into the tray 10. The separate well 22, as shown in FIG. 2A, has a hanger 40, and a well outer surface 24. The second separate well 22 can be oriented with the well bottom closed end 23 oriented downward, and can then be placed into a well aperture 20. FIG. 2A also shows the feet 25 of the tray 10 and well apertures 20 having raised rims 21. Well apertures 20 are also shown.

FIG. 2B shows a well 22 having a top opening 26, a bottom closed end 23, and at least one side wall 28 defining a well enclosure 19. The well 22 of FIG. 2B has an outer surface 24 and an inner surface 27. In embodiments, wells have a hanger 40. Wells 22 may have any shape. For example, wells 22 may be square, inverse pyramidal, conical, square, trapezoidal, hexagonal, octagonal, or any shape. Wells 22 may be, for example, Transwell® inserts available from Corning Incorporated. Inner surfaces 27 may be chamfered or may have a rounded or tapered top opening 26. The wells 22 may have smooth continuous inner surfaces 27 from the top opening of the well 22, or the well inner walls 27 may have a discontinuous profile. Wells 22 may have well side openings 29. In embodiments, well side openings 29 are provided to allow access to a reservoir (whether the reservoir is a well of a multi-well plate or a single reservoir) or a well of a multi-well plate below the removable well 22. For example, a pipette tip, inserted into a well and through the well side openings 29, may be extended to the reservoir 35 containing the well 22, to add or remove liquid from the reservoir 35.

Wells have a bottom 23 which has a bottom inner surface 46. Bottom surface 46 may be flat or may be shaped. For example, bottom surface 46 may be rounded, conical, inverse pyramidal, tapered, or any other shape suitable for supporting cells in culture. Well side walls 24 may be straight or angled. Well bottom inner surface 46 may have a treatment. For example, well bottom inner surface 46 may be formed from gas permeable, liquid impermeable, gas permeable and liquid permeable, track etched or porous material. Bottom inner surface 46 may have structures to encourage particular cells to grow. For example, the well bottom inner surface 46 may be treated so that adherent cells bind to the well bottom 46 (cell adherent treatment or cell binding treatment). Examples of cell adherent treatment include plasma treatment, coating with a cell binding material such as an extracellular matrix material, coating with a synthetic material such as Synthemax® available from Corning Incorporated, or other treatment. Or, well bottom 23 may be treated with a non-binding coating so that cells do not attach to the surface of the well bottom (cell non-binding treatment). Examples of cell non-binding treatment include coating the surface of the well bottom with Ultra Low Attachment material, ULA, available from Corning Incorporated. Or, the well bottom inner surface 46 may have micro-cavities to encourage cells to grow as clusters or cells, to form spheroids or tissues or tissue-like multicellular groups or clumps of cells. Microcavities are described as concave arcuate surfaces in, for example, US2014/0322806, incorporated herein in its entirety by reference. Or, well bottom surface 46 may have any combination of these treatments. In embodiments, well bottom surface 46 may have a treatment comprising gas permeable membrane, porous membrane, cell adherent treatment, cell non-binding treatment, microcavities, or a combination.

In embodiments, wells 22 have a hanger 40 (see, for example, FIG. 2). The hanger 40 is a structure such as, for example, a flange or an annular ring, extending from the outer surface 24 of the wells 22. The hanger 40 may be continuous all the way around the well opening 26, or the hanger 40 may be discontinuous, having structures that form protrusions from the top of well, but not a complete ring or annular structure. In embodiments, when the removable well 22 is inserted into a well aperture 20, there is a space 101 between the deck 13 of the tray 10 and the hanger 40 of the well 22 to allow the user to insert a tool, such as forceps, between the deck 13 of the tray 10 and the hanger 40 so that the hanger 40 is easily grasped by a user, and so that the hanger 40 can be easily inserted into, removed from, adjusted in, or manipulated in the well aperture 20. This space 101 between the deck 13 of the tray 10 and the hanger 40 of the well 22 can be accomplished in several ways, as described in FIGS. 3A-3E.

FIGS. 3A-3E show different embodiments of features which provide a space between the deck 13 of the tray 10 and the hanger 40 of the removable well 22. In embodiments, as shown in FIG. 3A, the cell culture well tray assembly of each well aperture 20 has a raised rim 21 extending upwardly from the top surface 11 of the deck 13. In this embodiment, the hanger 40 of well 22 is structured to engage with the top surface 16 of the raised rim 21 of the well apertures 20 so that the hanger 40 of the well 22 rests on the raised rim 21 of the well aperture when the well 22 is placed into a well aperture 20. The raised rim 21 of well apertures 20 (also shown in, for example, FIG. 2) serves as a positioning element to provide a space 101 between the removable well 22 and the deck 13 of the tray 10. This space 101 allows a user to insert forceps or tweezers between the deck 13 of the tray 10 and the removable well 22, or the hanger 40 of the removable well 22 to grasp the hanger 40 with forceps or tweezers, and remove an individual well 22. In embodiments, when the hanger 40 of the well 22 rests on the raised rim 21 of the well aperture 20, there is enough space between the bottom of the hanger 40 and the deck 13 of the tray 10, so that a user can insert forceps, tweezers, or a finger underneath the hanger 40 of the well 22 so that the well can be maneuvered or removed from the tray 10. Said another way, a user can grasp the hanger 40 of the well 22 because the hanger 40 does not sit flush against the deck 13 of the tray 10, but rather is raised from the deck 13 of the tray 10, or has a space 101 allowing a user to access and grasp the well 22 by the hanger 40. The well 22 can then be moved to another well aperture 20, removed from the tray 10, or otherwise manipulated. A tray 10 having a raised rim 21 of the well aperture 20 may be present alone to provide a space 101 between the deck 13 of the tray 10 and the hanger 40 of the well 22 or may be used in conjunction with angled sidewalls 31 of wells 22 as shown in FIG. 3B, a collar 45 as shown in FIG. 3C, angled well sidewalls 31 as shown in FIG. 3B, or a seat 105 in the well aperture 20 as shown in FIGS. 3D and 3E, or combinations of these features, to provide a space 101 between the deck 13 of the tray 10 and the hanger 40 of the well 22.

In additional embodiments, as shown in FIG. 3B, the well aperture 20 of the tray 10 has an angled sidewall 31. The angled sidewall 31 of the well aperture 20 catches against sidewall 28 of the well 22, so that the well top opening 26 extends above the top surface 11 of the deck 13 to create a space between the hanger 40 of the well 22 and the deck 13 of the tray 10. As shown in FIG. 3B, the well 22 may also have a chamfered or angled sidewall 28. When the well 22 having an angled or chamfered sidewall 28 is placed into an angled sidewall 31 of a well aperture 20, the well 22 extends into the well aperture 20 until the diameter of the chamfered well wall or walls 28 prevents the angled well 22 from extending further into the well aperture 20. In this embodiment, the chamfered or angled side wall 28 of the well 22 stops the hanger 40 from sitting flush against the deck 13 of the tray 10 and provides a space 101 between the deck 13 of the tray 10 and the hanger 40 of the well 22. In embodiments, the side wall 28 of the well 22 may be angled or not. In either embodiment, a space 101 is provided between the top surface 11 of the deck 13 and the hanger 40 of the well 22 when the removable well 22 is inserted into the well aperture 20. In embodiments, the angled sidewall 28 of the well 22 may be used alone to provide a space between the deck 13 of the tray 10 and the hanger 40 of the well 22. In additional embodiments, well apertures 20 having angled sidewalls 31 may be combined with a tray 10 having a raised rim 21 of the well aperture 20 or may be used in conjunction with a collar 45 as shown in FIG. 3C, angled well sidewalls 31 as shown in FIG. 3B, or a seat 105 in the well aperture 20 as shown in FIGS. 3D and 3E, or combinations of these features, to provide a space 101 between the deck 13 of the tray 10 and the hanger 40 of the well 22.

In additional embodiments, as shown in FIG. 3C, the well may have a collar 45 or spacer below the hanger 40. In this embodiment, when the removable well 22 is inserted into a well aperture 20, collar 45 sits flush against the well aperture, and provides a space 101 between the deck 13 of the tray 10 and the hanger 40 of the well. A well having a collar 45 as shown in FIG. 3C may be used alone to provide a space 101 between the deck 13 of the tray 10 and the hanger 40 of the well 22. Or, in embodiments, collar 45 may be employed in conjunction with a well aperture 20 having a raised rim 21 as shown in FIG. 3A, angled well apertures as shown in FIG. 3B, angled well sidewalls 31 as shown in FIG. 3B, or a seat 105 in the well aperture 20 as shown in FIGS. 3D and 3E, or combinations of these features, to provide a space 101 between the deck 13 of the tray 10 and the hanger 40 of the well 22.

In additional embodiments, as shown in FIG. 3D, well aperture 20 has a seat 105, which is an indent or a ridge or shelf present in the well aperture 20 in the wall of the aperture, structured to accommodate a removable well 22. In this embodiment, each well aperture 20 comprises a seat 105 and the removable well 22 has a collar 45 whose height is greater than the depth 110 of the seat 105. The depth 110 of the seat 105 can be measured from the top of the seat 105 to the top surface 11 of the deck 13. Or, as shown in FIG. 3E, seat 105 may provide a reduced diameter opening of well apertures 20, allowing an angled sidewall 28 of well 22 to rest against seat 105, presenting hanger 40 of well above the top surface 11 of deck 13 of tray 10 forming space 101. Seat 105 provides a barrier for the removable wells 22, so that sidewalls 28 of removable wells 22 engage against the seat 105 to ensure that the removable wells 22 are placed and contained within well apertures 20 in an appropriate and acceptable manner. For example, seat 105 may provide a rim upon which removable well 22 sits, allowing collar 45 (collar 45 has a height that is greater than the depth of the well 22 to extend above the top surface 11 of deck 13 of tray 10, presenting hanger 40 of well 22 above the top surface 11 of deck 13 of tray 10, providing space 101. Or, seat 105 may provide a reduced diameter opening of well apertures 20, allowing an angled or chamfered sidewall 28 of well to rest against seat 105, presenting hanger 40 of well above the top surface 11 of deck 13 of tray 10, providing space 101. By presenting hanger 40 of well above top surface 11 of deck 13 of tray 10, a space 101 is provided between the deck 13 and the hanger 40 of the well 22, allowing a user to insert forceps or tweezers between the deck 13 and the hanger 40 of the well 22, grasp the hanger 40, and remove the removable well 22. In this embodiment, each aperture has a seat 105 and the removable well 22 has a collar 45 whose height is greater than the depth of the seat 105, or an angled side wall 104 which catches against the seat 45 as the removable well 22 is placed into the well aperture 20 of the tray 10. In this embodiment, the seat 105, as described may be used to provide a space 101 between the deck 13 of the tray 10 and the hanger 40 of the well 22. In additional embodiments, the seat 105 may be provided in combination with a well 22 having a collar 45 as shown in FIG. 3C, in combination with a well aperture 20 having a raised rim 21 as shown in FIG. 3A, in combination with angled sidewalls 31 of wells 22 as shown in FIG. 3B, or in combination with angled well sidewalls 28 as shown in FIG. 3B, or in combination with more than one of these features, to provide a space 101 between the deck 13 of the tray 10 and the hanger 40 of the well 22.

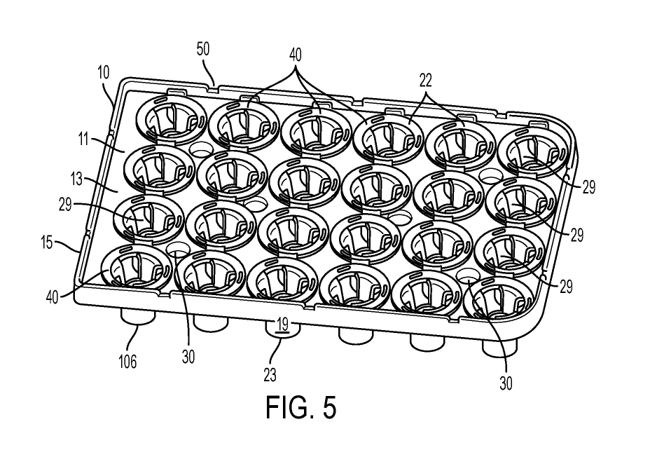

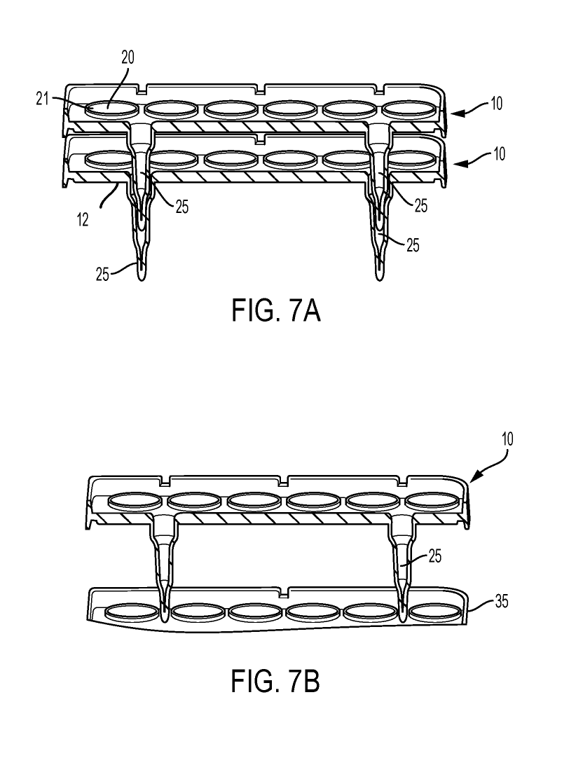

In embodiments, the well 22, placed in the well aperture 20 is still able to move in relation to the well aperture 20. For example, in embodiments, the well 22, placed into a well aperture 20, is free to rotate in relation to the well aperture 20 (and the tray 10), and is able to move up or down in relation to the well aperture 20 (or the tray 10). For example, when a well 22 is placed into a well aperture 20, and the tray 10 rests in a cell culture media reservoir 35 (see, for example, FIG. 6 and FIG. 7) the well 22 can slowly fill with media, as the well sinks into the cell culture media reservoir 35, all while the well 22 is placed within a well aperture 20. This allows the contents of the well 22 to equilibrate with the contents of the cell culture media reservoir 35. Wells 22 may already contain cells and/or cell culture media, when placed into a tray 10. In some instances, it may be that wells 22 already containing cells and/or cell culture media may have different amounts of liquid in each well when introduced into apertures 20 in the tray 10. By allowing the wells 22 to move within the well apertures 20, wells, each containing a different amount of liquid, can be placed through a well aperture 20 and may be placed into a reservoir 35 containing cell culture media, and each well can equilibrate with the level of media in the reservoir 35. In this way, each well 22 may be contained in the tray 10 and may reside in each well aperture 20 but may each have a different height in relation to the deck 13 of the tray. Wells 22 shown in FIGS. 3A-3E may have side openings 29 (not shown, but see FIG. 5, FIG. 2B or FIG. 6, for example).

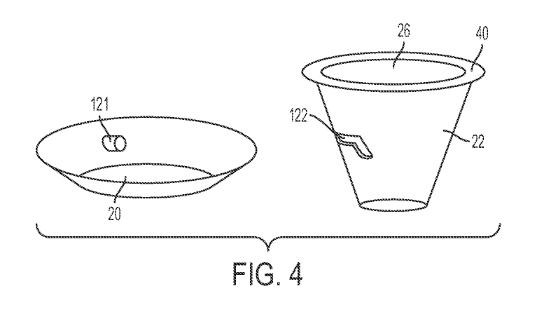

As shown in FIG. 4, in additional embodiments, the well aperture 20 or deck 13 and the well aperture 20 may have complimentary locking features 121, 122 to allow wells 22 to be releasably locked into place in the well apertures 20 of the tray 10, so that the wells 22 do not move in relation to the tray 10. As shown in FIG. 4, locking feature 121 in an angled well aperture 20, can be structured to engage with locking feature 122 in an angled sidewall 28 of well 22 (see FIG. 3B). Complimentary locking features (121, 122) may be provided in any of the embodiments shown in FIGS. 3A-3E. By locking the wells 22 in place, it is possible to ensure that the wells 22, having well side openings 29, are oriented in the well apertures 20 so that the well side openings 29 are all in the same location. When wells 22 are oriented in a locked position, a user can access the reservoir through well side openings 29 that are all aligned in the same orientation. In embodiments, this orientation of the wells 22, through the use of a locking mechanism, may enable a user to more predictably and rapidly access and manipulate the contents of a reservoir manually, using a single pipette tip or a multi-tip pipettor, or using robotic handling. As will be understood by those of skill in the art, locking features (121, 122) may be on the raised rim 21, on the seat 105, on the angled wall 31 or near the well apertures 20. In addition, while a male lock feature 121 is shown in FIG. 4 in the well aperture and the female lock feature 122 is shown on the sidewall of the well, the complimentary locking features (121, 122) may be placed in either location. In embodiments, the hanger may comprise a lock feature (121, 122) which may lock against a complimentary lock feature in the well aperture or the sidewall of the well.

FIG. 5 is a perspective view of an embodiment of a tray 10 containing removable wells 22. FIG. 5 shows a cell culture well tray 10 structured to fit on top of a reservoir plate 35, having a top surface 11 and an opposing bottom surface (not shown) defining a deck 13, a plurality of well apertures 20 in the deck 13; containing a plurality of removable wells 22, structured to fit through a well aperture 20 and extend below the bottom surface of the deck 13 of the tray 10, each well having a well enclosure 19 for containing cell culture material and a hanger 40 wherein when the removable well 22 is inserted into a well aperture 20, there is a space 101 between the deck 13 of the tray 10 and the hanger 40 of the well 22.

FIG. 5 also illustrates that removable wells 22 can be placed into the tray 10 in the same orientation. That is, wells 22, having side openings 29, can be placed into tray 10 in a way that orients the side openings 29 so that they are aligned. If the reservoir is in the form of a multi-well plate, so that each well 22 is inserted into a single well of a multi-well plate, the reservoir of the single well of the multi-well plate is accessed through the well side opening 29. If the wells 22 are aligned so that the side openings 29 are oriented in the same direction, robotic pipetting, which requires good alignment between the wells 22, is possible. Locking features (121, 122) may be placed in the same location within each aperture 20 and on each well 22 so that wells 22 may be locked into place in a tray 10 so that the side openings 29 are all oriented in the same direction and locked in place.

FIG. 5 also illustrates fill apertures 30. Fill apertures 30 are apertures through the deck 13 of the tray 10 that allow a pipette, inserted through the fill aperture 30 to access the contents of the reservoir below the wells, when the reservoir is a single reservoir. In embodiments, the cell culture well tray has at least one fill aperture in the deck.

FIG. 6 is an exploded view of a tray containing wells and a reservoir, according to embodiments. FIG. 6 illustrates a cell culture well tray assembly having a multi-well reservoir plate 35 for containing cell culture media, a removable tray 10 structured to fit on top of the plate 35, having a top surface 11 and an opposing bottom surface defining a deck 13, and a plurality of well apertures 20 in the deck 13; and at least one removable well 22, structured to fit through a well aperture 20 and extend below the bottom surface of the deck 13 of the tray 10, each well 22 having a well enclosure 19 for containing cell culture material and a hanger 40; wherein when the removable well 22 is inserted into a well aperture 20, there is a space 101 between the deck 13 of the tray 10 and the hanger 40 of the well 22. FIG. 6 illustrates the assembly with a multi-well plate providing the reservoir plate 35, having a separate well to contain each removable well 22, but the reservoir may be a single-reservoir plate 35 having a single shared reservoir for the wells. In this embodiment, the reservoir plate is a multi-well plate, each well of the multi-well plate structured to individually contain media and a cell culture well. The reservoir plate 35 is structured to receive at least a portion of the at least one well 22 extending below the bottom surface of the deck 13 of the tray 10. FIG. 6 also illustrates a lid 200, structured to fit on the tray. Tray 10 has feet 25 structured to fit between the wells of a multi-well reservoir 35 and to support tray in reservoir 35. See FIG. 7B.

As shown in FIG. 7A, in embodiments, tray 10 has a plurality of feet 25 extending from the opposing bottom surface 12 of the deck 13 wherein the feet 25 are structured to stack inside the feet 25 of another tray for storage and shipping (as shown in FIG. 7A) or to insert between wells of a multi-well plate reservoir (as shown in FIG. 7B) or to support the tray 10 when it is placed in a single reservoir, as shown in FIG. 8. FIG. 8 is a side cut-away view of an embodiment of a tray 10, containing removable wells 22, in a single reservoir 35. FIG. 8 also illustrates a lid 200. Feet 25 allow the tray 10, containing wells 22, to be placed into a reservoir 35 of cell culture media without the bottoms of the wells touching the bottom of the reservoir 35. This allows the bottom surface 46 of the well bottom 23 to be in bathed in cell culture media. Feet 25 also allow the tray 10 to be removed from a reservoir 35, such as a single reservoir (as shown in FIG. 8) and rest on a surface without contact between the bottom of the wells 22 and the surface. This reduces the risk of contamination.

In further aspects of the disclosure, it is specifically contemplated that any two or more aspects, embodiments, or features disclosed herein may be combined for additional advantage.

As used herein, the singular forms “a,” “an” and “the” include plural referents unless the context clearly dictates otherwise. Thus, for example, reference to a “notch” includes examples having two or more such “notches” unless the context clearly indicates otherwise

The term “include” or “includes” means encompassing but not limited to, that is, inclusive and not exclusive.

“Optional” or “optionally” means that the subsequently described event, circumstance, or component, can or cannot occur, and that the description includes instances where the event, circumstance, or component, occurs and instances where it does not.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It is also noted that recitations herein refer to a component being “configured” or “adapted to” function in a particular way. In this respect, such a component is “configured” or “adapted to” embody a particular property, or function in a particular manner, where such recitations are structural recitations as opposed to recitations of intended use. More specifically, the references herein to the manner in which a component is “configured” or “adapted to” denotes an existing physical condition of the component and, as such, is to be taken as a definite recitation of the structural characteristics of the component.

While various features, elements or steps of particular embodiments may be disclosed using the transitional phrase “comprising,” it is to be understood that alternative embodiments, including those that may be described using the transitional phrases “consisting” or “consisting essentially of,” are implied. For example, implied alternative embodiments to a microplate comprising polypropylene include embodiments where a microplate consists of polypropylene and embodiments where a microplate consists essentially of polypropylene.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A cell culture well tray assembly comprising:
a reservoir plate;
a removable tray structured to fit on top of the reservoir plate, having a top surface and an opposing bottom surface defining a deck, and a plurality of well apertures in the deck; and
at least one removable well, structured to fit in a well aperture and extend below the bottom surface of the deck of the tray, each well having a well enclosure for containing cell culture material and a hanger;
wherein the hanger has a top surface and a bottom surface;
wherein when the removable well is inserted into a well aperture, there is a space between the top surface of the deck of the tray and the bottom surface of the hanger of the well so that the bottom surface of the hanger is raised above the top surface of the deck of the tray.

2. The cell culture well tray assembly of claim 1 wherein each well aperture comprises a raised rim extending upwardly from the top surface of the deck.

3. The cell culture well tray assembly of claim 1 wherein the removable well comprises an angled sidewall which catches against a well aperture to create a space between the hanger of the well and the deck of the tray.

4. The cell culture well tray assembly of claim 1 wherein each aperture further comprises a seat and wherein the removable well comprises a tall side wall whose height is greater than the depth from the top of the seat to the top surface of the deck.

5. The cell culture well tray assembly of claim 2 wherein at least one of the raised rim and the well comprises a lock feature to releasably lock the well in place in the well aperture.

6. The cell culture well tray assembly of claim 1 wherein the tray further comprises a plurality of feet extending from the bottom surface of the deck wherein the feet are structured to insert between wells of a multi-well plate.

7. The cell culture well tray assembly of claim 1 further comprising at least one fill aperture in the deck.

8. The cell culture well tray assembly of claim 7 wherein the hanger comprises a lock feature.

9. The cell culture well tray assembly of claim 1 wherein the reservoir plate is structured to receive at least a portion of the at least one well extending below the bottom surface of the deck of the tray.

10. The cell culture well tray assembly of claim 9 wherein the reservoir plate comprises a single reservoir.

11. The cell culture well tray assembly of claim 9 wherein the reservoir plate is a multi-well plate, each well of the multi-well plate structured to individually contain media and a cell culture well.

12. The cell culture well tray assembly of claim 1 further comprising a lid structured to fit on the tray.

13. The cell culture well tray assembly of claim 1 wherein the tray further comprises a raised bezel around the outer periphery of the deck.

14. The cell culture well tray assembly of claim 1 any one of wherein the removable well comprises a well bottom inner surface which comprises a treatment.

15. The cell culture well tray assembly of claim 14 wherein the treatment comprises gas permeable membrane, porous membrane, cell adherent treatment, cell non-binding treatment, microcavities, or a combination.
